(19) United States
(12) Patent Application Publication
(10) Pub. No.: US 2014/0276774 A1
(43) Pub. Date: Sep. 18, 2014

(54) TISSUE DISSECTION DEVICE AND RELATED METHODS OF USE

(71) Applicant: BOSTON SCIENTIFIC SCIMED, INC., Maple Grove, MN (US)

(72) Inventors: Samuel RAYBIN, Marlborough, MA (US); Paul SMITH, Smithfield, RI (US); Naroun SUON, Lawrence, MA (US)

(73) Assignee: BOSTON SCIENTIFIC SCIMED, INC., Maple Grove, MN (US)

(21) Appl. No.: 14/210,044

(22) Filed: Mar. 13, 2014

Related U.S. Application Data
(60) Provisional application No. 61/784,531, filed on Mar. 14, 2013.

(51) Int. Cl. A61B 18/14

(52) U.S. Cl. CPC A61B 18/14
USPC 606/39

(57) ABSTRACT

Embodiments of the present disclosure include a method for treating tissue. The method may include delivering a medical device through a channel of an endoscopic device, wherein the medical device may include an elongate member, a cap coupled to a distal end of the elongate member and having a cavity, and an electrically-conductive tool extending into the cavity of the cap. The method may also include extending a portion of the medical device out of the channel of the endoscopic device and to the tissue, drawing a portion of the tissue into the cavity of the cap, electrically activating the tool to pierce the portion of the tissue, and cutting the tissue by moving the medical device along a plane of the tissue.

### CROSS-REFERENCE TO RELATED APPLICATIONS

This application claims the benefit of priority from U.S. Provisional Application No. 61/784,531, filed on Mar. 14, 2013, the entirety of which is incorporated by reference herein.

### FIELD OF THE DISCLOSURE

Embodiments of the present disclosure include medical devices and related methods for dissecting tissue.

### BACKGROUND OF THE DISCLOSURE

Organ walls are composed of several layers: the mucosa (the surface layer), the submucosa, the muscularis (muscle layer), and the serosa (connective tissue layer). In gastrointestinal, colonic, and esophageal cancer, e.g., small polyps or cancerous masses may form along the mucosa and often extend into the lumens of the organs. Conventionally, this condition is treated by cutting out a portion of the affected organ wall. This procedure, however, may cause discomfort to patients, and pose health risks. Recently, physicians have adopted a minimally invasive technique called endoscopic mucosal resection (EMR), and another called endoscopic submucosal dissection (ESD), which removes the cancerous or abnormal tissues (e.g., polyps), keeping the walls intact. EMR may also assist in removing any undesired tissue, even if such tissue is not abnormal or diseased.

EMR and ESD are generally performed with an endoscope, which may be a long, narrow elongate member optionally equipped with a light, imaging device, and other instruments and defining one or more lumens extending from a proximal to a distal end of the elongate member. During these procedures, the endoscope is passed down the throat or guided through the rectum to reach an undesired tissue, such as a polyp, in an affected organ. The distal end of the endoscope, typically equipped with a hood or a cap carrying dissecting tools is guided towards the undesired tissue. The undesired tissue may then be drawn into the hood. This may be achieved by applying suction through working channel extending along the lumen, or by retracting a retraction tool that is extendable from the endoscope. When the undesired tissue is sufficiently drawn into the hood, the dissecting tool may dissect portions of the tissue or resect target tissue from the organ wall. Subsequently, the excised tissue may be extracted for examination, biopsy, or disposal.

In some applications, however, difficulties in visualizing and/or controlling the dissecting tool during the dissection operation may arise. Accordingly, there exists a need for an improved dissection device and method for providing improved visualization and control.

### SUMMARY OF THE DISCLOSURE

In accordance with an embodiment, a method for treating tissue may include delivering a medical device through a channel of an endoscopic device, wherein the medical device may include an elongate member, a cap coupled to a distal end of the elongate member and having a cavity, and an electrically-conductive tool extending into the cavity of the cap. The method may also include extending a portion of the medical device out of the channel of the endoscopic device and to the tissue, drawing a portion of the tissue into the cavity of the cap, electrically activating the tool to pierce the portion of the tissue, and cutting the tissue by moving the medical device along a plane of the tissue.

Various embodiments of the disclosure may include one or more of the following aspects: moving the medical device relative to the channel of the endoscopic device to cut the tissue, fixing the medical device to the endoscopic device and moving the endoscopic device to cut the tissue, applying suction through the elongate member of the medical device to draw the portion of the tissue through an opening of the cap and into the cavity of the cap, moving the medical device along the plane of the tissue while suction is applied through the elongate member, electrically activating the tool before the portion of the tissue is drawn into the cavity of the cap, electrically activating the tool after the portion of the tissue is drawn into the cavity of the cap, wherein the tool may include a tip disposed in the cavity of the cap, wherein the tip may be positioned substantially perpendicular to an opening of the cap, wherein the tool may extend through a lumen of the elongate member, wherein the tool may be disposed within a wall of the elongate member, wherein the tool may be fixed from movement relative to the elongate member, wherein the tool may be retractable relative to the elongate member, moving the tool relative to the elongate member to cut the tissue, wherein the endoscopic device may include a distal face, moving the medical device distal the distal face of the endoscopic device to cut the tissue, and wherein the endoscopic device may include an imaging device positioned at the distal face.

In accordance with another embodiment, a method for treating tissue may include delivering a medical device through a channel of an endoscopic device, wherein the medical device may include an elongate member, a cap coupled to a distal end of the elongate member, the cap having an opening and a cavity, and an electrically-conductive tool extending into the cavity of the cap. The method may also include extending a portion of the medical device out of the channel of the endoscopic device to the tissue, applying suction through the elongate member of the medical device to draw a portion of the tissue through the opening and into the cavity of the cap, electrically activating the tool to pierce the portion of the tissue, and moving the medical device relative to the channel of the endoscopic device to cut the tissue.

Various embodiments of the disclosure may include one or more of the following aspects: moving the medical device along a plane of the tissue while suction is applied through the elongate member to cut the tissue, wherein the endoscopic device may include a distal face, and moving the medical device distal the distal face of the endoscopic device to cut the tissue.

In accordance with yet another embodiment, a method for treating tissue may include delivering a medical device through a channel of an endoscopic device, wherein the medical device may include an elongate member, a cap coupled to a distal end of the elongate member, the cap having an opening and a cavity, and an electrically-conductive tool extending into the cavity of the cap. The method may also include extending the cap of the medical device out of the channel of the endoscopic device to a target tissue, separating a first tissue layer from a second tissue layer of the target tissue by drawing the first tissue layer through the opening and into the cavity of the cap, electrically activating the tool to pierce the first tissue layer, and moving the medical device relative to the channel of the endoscopic device to cut the first tissue layer.

Various embodiments of the disclosure may include one or more of the following aspects: applying suction through the elongate member of the medical device to draw the first tissue layer into the cavity of the cap and moving the medical device along the first tissue layer while suction is applied through the elongate member to cut the first tissue layer.

In accordance with yet another embodiment, an endoscopic device may include an elongate member having a lumen extending between a proximal end and a distal end of the elongate member. The endoscopic device may also include a cap at the distal end of the elongate member. The cap may include a cavity in communication with the lumen, and a cap longitudinal axis that is offset relative to a longitudinal axis of the elongate member. The endoscopic device may also include an electrically-conductive tool extending into the cavity of the cap.

Various embodiments of the disclosure may include one or more of the following aspects: the cap further including a laterally-facing opening in communication with the cavity; the tool including a tip disposed in the cavity of the cap, a length of the tip being positioned substantially perpendicular to the laterally-facing opening; an entirety of the tip being contained within the cavity; the tip being extendable up to but not through the laterally-facing opening such that no portion of the tip can pass through the laterally-facing opening; the tool being disposed within a wall of the elongate member; the tool being fixed from movement relative to the elongate member; the tool being retractable relative to the cavity and being configured to move relative to the elongate member to cut a tissue; the elongate member extending from the proximal end to the distal end in a first direction, and the cap extending from the elongate member in both the first direction and in a second direction that is substantially perpendicular to the first direction; and the cap further including an opening that is substantially parallel to the cap longitudinal axis.

In accordance with yet another embodiment, an endoscopic device may include a securing mechanism configured to engage around an outer surface of an elongate member, and a sealing interface configured to form a fluid-tight seal with a distal face of the elongate member. The endoscopic device may also include a cavity that is distal to the distal face of the elongate member, and a laterally-facing opening in communication with the cavity.

Various embodiments of the disclosure may include one or more of the following aspects: the sealing interface including an aperture configured to communicate with an opening disposed in a distal face of the elongate member; and the laterally-facing opening and the aperture being transverse to one another.

In accordance with yet another embodiment, an endoscopic device may include an elongate member having a distal end and a distal face extending substantially perpendicular to a longitudinal axis of the elongate member, and a distal cap at the distal end of the elongate member. The distal cap may include a securing mechanism engaged around an outer surface of the elongate member, and a sealing interface configured to form a fluid-tight seal with the distal face. The distal cap may also include a cavity that is distal to the distal face, and a laterally-facing opening in communication with the cavity.

Various embodiments of the disclosure may include one or more of the following aspects: the distal face including an opening, and the sealing interface including an aperture in communication with the opening, wherein the aperture and the laterally-facing opening are transverse to one another; a tool disposed through the elongate member, wherein a tip of the tool is disposed in the cavity of the distal cap, and a length of the tip is positioned substantially perpendicular to the laterally-facing opening; an entirety of the tip being contained within the cavity; a light source and an imaging device, both the light source and the imaging device disposed on the distal face of the elongate member; the distal cap partially extending across the distal face of the elongate member such that the light source and the imaging device are not covered by the distal cap; wherein only the sealing interface of the distal cap is in contact with the distal face of the elongate member.

### BRIEF DESCRIPTION OF THE DRAWINGS

FIG. 1 is a schematic illustration of an endoscopic device, according to an exemplary disclosed embodiment;

FIG. 2 is a perspective view of a medical device for use with the endoscopic device of FIG. 1, according to an exemplary disclosed embodiment;

FIG. 3 illustrates a cross-sectional view of a distal cap for the medical device of FIG. 2, according to an exemplary disclosed embodiment; and

FIG. 4 is a perspective illustration of another endoscopic device, according to an exemplary disclosed embodiment.

### DETAILED DESCRIPTION

Reference will now be made in detail to exemplary embodiments of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an endoscopic device 100 and a medical device 1. When used herein, “proximal” refers to a position relatively closer to the exterior of the body or closer to an operator, such as a surgeon, using endoscopic device 100 or medical device 1. In contrast, “distal” refers to a position relatively further away from the operator using endoscopic device 100 or medical device 1, or closer to the interior of the body.

FIG. 1 illustrates a schematic view of an exemplary endoscopic device 100 of the present disclosure. Endoscopic device 100 may be used for dissecting and/or resecting polyps, lesions, or other unwanted tissue from the interior bodily walls or other anatomical structure of a patient, or for any other medical procedure. Endoscopic device 100 may include an elongate member 102 having a proximal end 104, a distal end 106, and a lumen 108 extending between proximal and distal ends 104, 106. Proximal end 104 may be coupled to an appropriate handle (not shown), while distal end 106 may include one or more openings in communication with lumen 108.

Endoscopic device 100 may be an endoscope or any other suitable introduction device or sheath adapted to be advanced into a body lumen. For example, endoscopic device 100 may include a guide tube, an endoscope, a guide sheath, a flexible member, and/or a catheter. In the illustrated embodiment, lumen 108 of elongate member 102 may include one or more channels (not shown), through which, the operator may introduce one or more medical devices to extend out of distal end 106 of elongate member 102. For example, during a tissue dissection or resection procedure, the operator may introduce a suction device into one channel and a cutting device, such as, for example, a snare loop, into another channel. Additionally, the operator may insert a light source, a camera, an injector, or a morcellator within the one or more channels. Because different implements may need to be inserted into the elongate member 102, the dimensions of its channels may vary. Some channels may have a larger diameter, while others may have a smaller diameter. Further, some channels may include permanently fixed devices, such as light sources or imaging devices extending to a distal face of elongate member 102, while other channels may allow temporary insertion and removal of medical devices, as the operator desires. Elongate member 102 may also be any known endoscopic device used, for example, for colonoscopy, resectoscopy, cholangioscopy, tissue dissection, or mucosal resection.

Moreover, elongate member 102 may be coated with lubricious materials and antibacterial agents to ease insertion into tight cavities and prevent infections, respectively. Further, portions of the elongate member 102 may include radiopaque materials to visualize the position of elongate member 102 within a patient's body.

FIG. 2 illustrates a perspective view of medical device 1 for use with endoscopic device 100, according to an exemplary embodiment. Dashed lines are included in FIG. 2 to help illustrate certain internal structures and components of medical device 1. Medical device 1 may be delivered through lumen 108 of endoscopic device 100 and to a target area to treat tissue. In some embodiments, medical device 1 may be configured to dissect tissue of a target area, as will be described in more detail below. As shown in FIG. 2, medical device 1 may include a distal cap 2, a proximal handle 3, and an elongate member 4 operably connected to distal cap 2 and proximal handle 3.

Elongate member 4 may include any suitable sheath, tube, flexible member, or catheter including a lumen 5 extending therethrough. Elongate member 4 may also include a proximal end 6 coupled to proximal handle 3, and a distal end 7 coupled to distal cap 2.

Handle 3 may be attached to elongate member 4 by, for example, welding or adhesives, or may be integrally formed with elongate member 4. Handle 3 may include one or more ports (not shown) to introduce one or more medical devices or tools into lumen 5 of elongate member 4. Moreover, a suction source or an irrigation source (not shown) may be coupled to a port of handle 3 to generate a suction force through lumen 5, or deliver a suitable irrigation fluid through lumen 5, respectively. Suitable control members, such as valves, may be associated with handle 3 to control the suction and/or irrigation through lumen 5. Handle 3 may also include a suitable steering mechanism (not shown) configured to steer and/or deflect distal end 7 of medical device 1.

Medical device 1 may also include a tool 8 extending through lumen 5 and to distal cap 2. A proximal end of tool 8 may be operably coupled to an electrical port (not shown) on handle 3. The electrical port in turn may be coupled to a suitable electrical source (not shown), and may be configured to deliver electrical energy from the electrical source to tool 8. Accordingly, and as will be described in more detail below, tool 8 may include any suitable electrically-conductive device configured to dissect and/or cut tissue, including, as examples, a wire, a needle, a blade, a knife, or a needle knife. It should be appreciated, however, that tool 8 is not limited to tissue dissection and/or cutting devices, and may include any other suitable instrument or tool for any medical procedure.

Distal cap 2 may be coupled to distal end 7 of elongate member 4 by any suitable means. In one embodiment, for example, distal cap 2 may be detachably coupled to distal end 7 by any suitable coupling technique, such as, for example, a snap-fit, a luer-lock, screw threading, and the like. In certain other embodiments, distal cap 2 may be permanently secured to distal end 7 by, for example, adhesives, welds, and the like, or may be integrally formed with elongate member 4.

It should also be appreciated that one or both of elongate member 4 and distal cap 2 may include features configured to minimize risk and damage to surrounding tissues while in use. For example, one or more sections of elongate member 4 and distal cap 2 may include appropriate geometrical configurations to reduce trauma and irritation to surrounding tissues, such as, for example, rounded or beveled terminal ends or faces. Moreover, the outer surfaces of distal cap 2 and elongate member 4 may include a suitable covering or coating to minimize trauma and damage to tissues. For example, the outer surfaces may include a layer of lubricious material to facilitate and ease the insertion of medical device 1 through a body lumen. Additionally, or alternatively, the outer surfaces may be coated with an antibacterial coating to inhibit bacterial growth in the body cavity.

Distal cap 2 may be a generally hollow member defining a cavity 9 and a laterally-facing opening 10 in communication with cavity 9. Accordingly, upon application of a suction force through lumen 5, target tissue may be drawn through opening 10 and into cavity 9. For example, a tissue plane, such as the mucosa, may be drawn into cavity 9, while an underlying tissue plane, such as the muscularis, remains intact. Tool 8 may then dissect the tissue drawn into cavity 9. Medical device 1 therefore may separate tissue planes and dissect one of the separated tissue planes.

As shown in FIG. 2, distal cap 2 may be coupled to elongate member 4 at an angled configuration. That is, distal cap 2 may have a cap longitudinal axis that is offset relative to a longitudinal axis of elongate member 4. Elongate member 4 may extend from a proximal end to a distal end in a first direction. Distal cap 2 may thus extend from elongate member 4 in both the first direction and in a second direction that is substantially perpendicular to the first direction. The angled relationship between distal cap 2 and elongate member 4 may ease the placement of distal cap 2 on a tissue plane, such as, for example, a mucosal wall, and may allow a greater surface area of distal cap 2 to be in contact with target tissue. In addition, opening 10 may be defined on distal cap 2 offset (i.e., angled) relative to a longitudinal axis of distal cap 2. The slanted opening 10 may also provide for a greater surface area of contact between distal cap 2 and the target tissue. The slant angle of opening 10 may vary based on, for example, the organ or the procedure. For example, a narrower body organ, such as the esophagus, may call for an opening 10 slanted at a greater angle than for wider body organs, such as the stomach. In other embodiments, opening 10 may be substantially perpendicular to the longitudinal axis of distal cap 2 to define a distally-facing opening 10. Still in further embodiments, opening 10 may be substantially parallel to the longitudinal axis of distal cap 2. Moreover, it should be appreciated that opening 10 may extend any suitable distance along the length of distal cap 2.

The shape of opening 10 may also vary, as desired. In some embodiments, distal cap 2 may include any suitable oblong-shaped opening 10. As shown in FIG. 2, for example, opening 10 may include a substantially teardrop shape. The oblong shape of opening 10 may improve tissue retention within cavity 9, and may also prevent lateral movement of tissue drawn into cavity 9 as medical device 1 is advanced and/or retracted. The shape of opening 10 may also vary depending on, for example, the cross-sectional shape of distal cap 2 and/or the slant angle of opening 10. In some embodiments, a square or rectangle-shaped distal cap 2 may define a rectangular opening 10. In certain other embodiments, a cylindrical distal cap 2 may define a circular or oval shaped opening 10.

Distal cap 2 may be formed of any suitable material. For example, distal cap 2 may be formed of rigid or semi-rigid materials, such as metals (including super elastic materials, such as nitinol), polymers, resins, or plastics. In some embodiments, a distal portion of distal cap 2 may be formed of a flexible material, while the remaining portions may be rigid. Alternatively, only a proximal portion may be formed of a flexible material. Flexible portions may improve the sealing of distal cap 2 with the contacting tissue and/or elongate member 4. Furthermore, it should be appreciated that a distal portion of distal cap 2 may be configured to articulate relative to a proximal portion of distal cap 2.

Distal cap 2 and/or elongate member 4 may also be optically clear, allowing an operator to visualize tool 8 and tissue positioned within and/or around distal cap 2. Moreover, a biocompatible material configured to minimize tissue irritation may be applied as a coating over the outer surface of distal cap 2. The coating may also prevent blood from depositing and adhering on the outer surface of distal cap 2. In addition, the coating may be a lubricious coating, which may help to facilitate insertion of distal cap 2 into a patient's body.

Furthermore, suitable markings, such as, for example, radiopaque or sono-reflective markings (not shown), may be applied to the outer surface of distal cap 2. These markings may facilitate the detection of a position and/or orientation of distal cap 2 within the patient's body, and an operator, with the aid of suitable imaging equipment, may track the path of distal cap 2 and avoid potential damage to sensitive tissues. In some embodiments, a portion or a substantial entirety of the periphery of opening 10 may include a radiopaque or sono-reflective marking.

Although not illustrated, it should also be appreciated that distal cap 2 may include a grate or any other suitable mechanism to control the volume of tissue drawn into cavity 9. For example, the grate may include a number of longitudinal ridges extending from an inner surface of cavity 9 to opening 10 at a given distance. The plane defined by the upper surface of the ridges may control the depth to which tissue may be drawn into cavity 9, which in turn may define the volume of tissue to be dissected.

In certain embodiments, and as shown in FIG. 2, tool 8 may extend through lumen 5 of elongate member 4 and cavity 9 of distal cap 2. It should be appreciated, however, that in other embodiments, tool 8 may be disposed within a wall defining elongate member 4 and a wall defining distal cap 2.

As alluded to above, tool 8 may be configured to dissect tissue drawn into cavity 9. To that end, tool 8 may include a tip 11 disposed in cavity 9. A length of tip 11 may be positioned substantially perpendicular to opening 10. As such, tissue drawn into cavity 9 through opening 10 may be pierced by tip 11. Tip 11 may pierce the drawn tissue when tool 8 (and thus tip 11) is electrically-activated. Additionally, or alternatively, tip 11 may include a sharp end to pierce the drawn tissue with or without tool 8 being electrically-activated. In some embodiments, tip 11 may extend through opening 10 and may include a portion external distal cap 2. In other embodiments, an entirety of tip 11 may be contained within cavity 9 at all times and may extend up to but not through opening 10. That is, in some embodiments, no portion of tip 11 can pass through opening 10. It should also be appreciated that a portion of tool 8 proximal to tip 11 may be coated or encased by a suitable insulating material. The insulating material may be incapable of conducting energy to shield and prevent the discharge of stray electrical energy from tool 8.

Tip 11 may then dissect tissue by moving tip 11 relative to the tissue. As tip 11 cuts through the tissue, a dissection path along the tissue may be formed. In some embodiments, tool 8 may be fixed from movement relative to medical device 1. That is, tool 8 may be secured to one or more of handle 3, elongate member 4, and distal cap 2. Accordingly, medical device 1 may be advanced and/or retracted relative to endoscopic device 100 while tissue is sucked into cavity 9 and tool 8 is electrically-activated. As a result, tip 11 may dissect the tissue along a desired path. In certain other embodiments, tool 8 may be movably disposed within lumen 5 of elongate member 4 and cavity 9 of distal cap 2. For example, tool 8 may be coupled to a suitable actuation member (not shown) on handle 3 that may be configured to advance and retract tool 8 relative to lumen 5 of elongate member 4. As such, in addition to medical device 1 being advanced and/or retracted relative to endoscopic device 100, tool 8 may be advanced and/or retracted relative to elongate member 4 such that tip 11 dissects tissue along a desired path.

An exemplary method for dissecting tissue with medical device 1 is described below. Endoscopic device 100 may be inserted through a natural opening of patient, such as, for example, the mouth or anus, to reach a desired internal anatomy, or may be inserted through a percutaneous incision.

Endoscopic device 100 may then be directed towards target tissue. The light source and the imaging device of endoscopic device 100 may allow the operator to visualize and identify the internal anatomy and target tissue distal endoscopic device 100. Once endoscopic device 100 reaches the target tissue, medical device 1 may be delivered through a channel of elongate member 102. Alternatively, endoscopic device 100 and medical device 1 together may be delivered to the target tissue.

Medical device 1 may be extended out of distal end 106 of elongate member 102, and distal cap 2 of medical device 1 may be advanced over the target tissue. It should also be appreciated that one or both of elongate member 102 of endoscopic device 100 and elongate member 4 of medical device 1 may be deflected or steered to position distal cap 2 of medical device 1 over the target tissue. Furthermore, medical device 1 may be manipulated and positioned relative to the target tissue while being visualized by the operator via the light source and imaging device of endoscopic device 100.

In some embodiments, tip 11 of tool 8 may be disposed within cavity 9 and electrically activated once distal cap 2 is positioned over the target tissue. The suction source operably coupled to medical device 1 may then be activated to draw the target tissue into cavity 9 of distal cap 2. Accordingly, as the tissue is drawn into cavity 9, the electrically-activated tip 11 may pierce and cut into the drawn tissue. More particularly, medical device 1 may separate a first tissue plane from a second tissue plane by drawing a portion of the first tissue plane into cavity 9 via suction. As the first tissue plane enters cavity 9 and contacts tip 11, tip 11 may pierce and cut into the first tissue plane.

In other embodiments in which tool 8 is movably disposed within lumen 5 of elongate member 4, tool 8 may be proximally retracted prior to positioning distal cap 2 over the target tissue. Tool 8 may be proximally retracted such that tip 11 is proximal opening 10. The suction source then may be activated to draw tissue into cavity 9 of distal cap 2. Once the tissue is sucked into cavity 9, tool 8 may be electrically activated, and the operator may distally advance tool 8, causing tip 11 to cut into the tissue.

FIG. 3 illustrates a cross-sectional view of medical device 1 dissecting a target tissue, according to an exemplary disclosed embodiment. In the exemplary embodiment of FIG. 4, tool 8 may be disposed within a wall 14 of elongate member 4 and a wall 15 of distal cap 2. Medical device 1 may separate a first tissue plane 12 from a second tissue plane 13 by drawing a portion of first tissue plane 12 into cavity 9 of distal cap 2 via suction. Once tip 11 of tool 8 initially pierces first tissue plane 12, medical device 1 may be advanced and/or retracted to create a dissection path along first tissue plane 12. Medical device 1 may be advanced and/or retracted (as illustrated by the arrow A in FIG. 4) while suction is applied onto first tissue plane 12 to keep first tissue plane 12 and second tissue plane 13 separated. Moreover, because medical device 1 may be advanced and/or retracted relative to distal end 106 of elongate member 102, dissection of first tissue plane 12 may be visualized through endoscopic device 100.

It should also be appreciated that one or both of medical device 1 and endoscopic device 100 may be further manipulated to resect a portion of target tissue. For example, medical device 1 and/or endoscopic device 100 may be maneuvered around a perimeter of a lesion on first tissue plane 12 as a portion of first tissue plane 12 is drawn into cavity 9 and tool 8 is electrically-activated. Accordingly, the lesion may be resected from first tissue plane 12, and suction from medical device 1 or another suitable medical device delivered through endoscopic device 100 may be employed to retrieve and remove the resected tissue portion. As discussed, medical device 1 may be maneuvered by distally advancing and/or proximally retracting medical device 1 relative to elongate member 102 of endoscopic device 100. Additionally, or alternatively, medical device 1 may be fixed from movement relative to endoscopic device 100 by, for example, a suitable locking mechanism, and endoscopic device 100 may be distally advanced and/or proximally retracted to maneuver medical device 1. Furthermore, one or both of elongate member 4 of medical device 1 and elongate member 102 of endoscopic device 100 may be deflected or steered to guide the dissection path in the lateral directions (i.e., to the left and to the right). Additionally, or alternatively, the operator may simply move medical device 1 and/or endoscopic device 100 in the lateral directions to guide the dissection path formed by medical device 1.

Medical device 1 may provide a number of features. For example, medical device 1 may provide improved visualization and control when dissecting a target tissue. Since medical device 1 may be manipulated relative to elongate member 102 of endoscopic device 100, the operator may view and control medical device 1 via the light source and the imaging device of endoscopic device 100. Accordingly, the improved visualization when operating medical device 1 may allow the operator to better control the cutting depth and accuracy of medical device 1 and distinguish between different types of tissue.

FIG. 4 illustrates a perspective view of an exemplary endoscopic device 200 of the present disclosure. Similar to endoscopic device 100, endoscopic device 200 may include elongate member 102. As shown in FIG. 4, elongate member 102 may include one or more light sources 201, an imaging device 202, and one or more channels 203, 204.

Endoscopic device 200 may also include a distal cap 300 coupled to distal end 106 of elongate member 102. Distal cap 300 may include a suitable securing mechanism 301, such as, for example, a strap, a band, a clip, or the like, engaged around an outer surface of elongate member 102. Distal cap 300 may be a generally hollow member defining a cavity 309 and a laterally-facing opening 310 in communication with cavity 309. Distal cap 300 may be distal to a distal face 205 of elongate member 102, and include a sealing interface 302 configured to form a fluid-tight seal with distal face 205. More particularly, sealing interface 302 may be positioned over an opening 210 of channel 203, and may include an aperture 303 substantially aligned with opening 210 of channel 203. Accordingly, channel 203 may be in communication with cavity 309 of distal cap 300. Aperture 303 may also be transverse to opening 310.

An appropriate suction device (not shown) may be operably coupled to endoscopic device 200 and may be configured to provide suction through channel 203. Upon application of a suction force through channel 203, target tissue may be drawn through opening 310 and into cavity 309. A suitable tool 80 for resection and/or dissection procedures, such as, for example, a wire loop, a snare loop, a band, a wire, or a knife, may then be delivered through channel 203 to cavity 309 of distal cap 300. Tool 80 may also be electrically-conductive, and may dissect the tissue drawn into cavity 309.

Distal cap 300 may be appropriately sized such that light sources 201 and imaging device 202 are not be obstructed by distal cap 300. That is, distal cap 300 may partially extend across distal face 205 of elongate member 102 such that light sources 201 and imaging device 202 are not covered by distal cap 300. Moreover, only sealing interface 302 of distal cap 300 may be in contact with distal face 205 of elongate member 102 and may be coupled around a periphery of opening 210. Accordingly, distal cap 300 may also provide improved visualization and control of tool 80 when dissecting a target tissue. Since distal cap 300 does not obstruct the path of light sources 201 and imaging device 202, the operator may more effectively view and control tool 80 during a dissection and/or resection procedure.

Any aspect set forth in any embodiment may be used with any other embodiment set forth herein. Every device and apparatus set forth herein may be used in any suitable medical procedure, may be advanced through any suitable body lumen and body cavity, and may be used to access tissue from any suitable body portion. For example, the apparatuses and methods described herein may be used through any natural body lumen or tract, including those accessed orally, vaginally, rectally, nasally, urethrally, or through incisions in any suitable tissue.

It will be apparent to those skilled in the art that various modifications and variations can be made in the disclosed systems and processes without departing from the scope of the invention. Other embodiments of the invention will be apparent to those skilled in the art from consideration of the specification and practice of the invention disclosed herein. It is intended that the specification and examples be considered as exemplary only. The following disclosure identifies some other exemplary embodiments.

What is claimed is:

1. A method for treating tissue, comprising:
delivering a medical device through a channel of an endoscopic device, wherein the medical device includes:
an elongate member;
a cap at a distal end of the elongate member and having a cavity; and
an electrically-conductive tool extending into the cavity of the cap;
extending a portion of the medical device out of the channel of the endoscopic device and to the tissue;
drawing a portion of the tissue into the cavity of the cap;
electrically activating the tool to pierce the portion of the tissue; and
cutting the tissue by moving the medical device along a plane of the tissue.

2. The method of claim 1, further comprising moving the medical device relative to the channel of the endoscopic device to cut the tissue.

3. The method of claim 1, further comprising fixing the medical device to the endoscopic device and moving the endoscopic device to cut the tissue.

4. The method of claim 1, further comprising applying suction through the elongate member of the medical device to draw the portion of the tissue through an opening of the cap and into the cavity of the cap.

5. The method of claim 4, further comprising moving the medical device along the plane of the tissue while suction is applied through the elongate member.

6. The method of claim 1, further comprising electrically activating the tool before the portion of the tissue is drawn into the cavity of the cap.

7. The method of claim 1, further comprising electrically activating the tool after the portion of the tissue is drawn into the cavity of the cap.

8. The method of claim 1, wherein the tool includes a tip disposed in the cavity of the cap, wherein the tip is positioned substantially perpendicular to an opening of the cap.

9. The method of claim 8, wherein the tool extends through a lumen of the elongate member.

10. The method of claim 8, wherein the tool is disposed within a wall of the elongate member.

11. The method of claim 8, wherein the tool is fixed from movement relative to the elongate member.

12. The method of claim 8, wherein the tool is retractable relative to the elongate member, and further comprising moving the tool relative to the elongate member to cut the tissue.

13. The method of claim 1, wherein the endoscopic device includes a distal face, and further comprising moving the medical device distal the distal face of the endoscopic device to cut the tissue.

14. The method of claim 13, wherein the endoscopic device includes an imaging device positioned at the distal face.

15. A method for treating tissue, comprising:
delivering a medical device through a channel of an endoscopic device, wherein the medical device includes:
an elongate member;
a cap at a distal end of the elongate member, the cap having an opening and a cavity; and
an electrically-conductive tool extending into the cavity of the cap;
extending a portion of the medical device out of the channel of the endoscopic device to the tissue;
applying suction through the elongate member of the medical device to draw a portion of the tissue through the opening and into the cavity of the cap;
electrically activating the tool to pierce the portion of the tissue; and
moving the medical device relative to the channel of the endoscopic device to cut the tissue.

16. The method of claim 15, further comprising moving the medical device along a plane of the tissue while suction is applied through the elongate member to cut the tissue.

17. The method of claim 15, wherein the endoscopic device includes a distal face, and further comprising moving the medical device distal the distal face of the endoscopic device to cut the tissue.

18. A method for treating tissue, comprising:
delivering a medical device through a channel of an endoscopic device, wherein the medical device includes:
an elongate member;
a cap at a distal end of the elongate member, the cap having an opening and a cavity; and
an electrically-conductive tool extending into the cavity of the cap;
extending the cap of the medical device out of the channel of the endoscopic device to a target tissue;
separating a first tissue layer from a second tissue layer of the target tissue by drawing the first tissue layer through the opening and into the cavity of the cap;
electrically activating the tool to pierce the first tissue layer; and
moving the medical device relative to the channel of the endoscopic device to cut the first tissue layer.

19. The method of claim 18, further comprising applying suction through the elongate member of the medical device to draw the first tissue layer into the cavity of the cap.

20. The method of claim 19, further comprising moving the medical device along the first tissue layer while suction is applied through the elongate member to cut the first tissue layer.
